SEQUENCE LISTING (5 sequences)

SEQ ID NO: 1 - protein, 6 aa, synthetic construct
GRGDSP

SEQ ID NO: 2 - protein, 8 aa, synthetic construct
  MOD_RES 8: note = Amidation
RFRFRFRF

SEQ ID NO: 3 - protein, 8 aa, synthetic construct
  MOD_RES 8: note = amidation
  SITE 1: note = N-((4-benzoyl)benzoyl) 6-amino hexanoyle
RFRFRFRF

SEQ ID NO: 4 - protein, 8 aa, synthetic construct
  MOD_RES 8: note = amidation
RIRIRIRI

SEQ ID NO: 5 - protein, 8 aa, synthetic construct
  MOD_RES 8: note = amidation
  SITE 1: note = N-((4-benzoyl)benzoyl) 6-amino hexanoyle
RIRIRIRI

(19) United States
(12) Patent Application Publication
(10) Pub. No.: US 2026/0271922 A1
(43) Pub. Date: Sep. 17, 2026

(54) METHOD FOR FUNCTIONALISING A SOLID SUBSTRATE WITH A PEPTIDE CONJUGATE

(71) Applicant: GENEPEP, SAINT JEAN DE VEDAS (FR)

(72) Inventors: SAÏD JEBORS, MONTPELLIER (FR); KARINE PUGET, SAINT JEAN DE VEDAS (FR)

(21) Appl. No.: 19/471,827

(22) PCT Filed: Apr. 3, 2024
(86) PCT No.: PCT/EP2024/059023

§ 371 Date: Oct. 2, 2025

(30) Foreign Application Priority Data
Apr. 4, 2023 (FR) FR2303338

(51) Int. Cl. A01N 25/34; A01P 1/00; C07K 7/06; C07K 17/08

(52) U.S. Cl. CPC A01N 25/34; A01P 1/00; C07K 7/06; C07K 17/08

(57) ABSTRACT

The present invention relates to a method for functionalising a solid support with a peptide conjugate comprising a benzophenone anchor head, a spacer arm and a peptide fragment. The invention also relates to a solid substrate that can be obtained by a functionalisation method according to the invention, as well as to the use of the solid substrate according to the invention.

### TECHNICAL FIELD

The present invention relates to the field of functionalization of surfaces, such as textile or plastic surfaces, with peptides, notably antimicrobial peptides.

### TECHNOLOGICAL BACKGROUND

The medical industry is faced with many challenges. Among the main ones, mention may be made of infection control and reducing the spread of microorganisms such as fungi, bacteria, and viruses. Some of these microorganisms can attach themselves to surfaces and form biofilms. Beyond the medical field, biofilms also represent a threat in the food processing industry. Biofilms have conventionally been treated with antibiotics, but this widespread use of antibiotics has contributed to the emergence of strains that have developed resistance to antibiotics. It is now therefore necessary to develop alternative methods to the use of disinfectants and antibiotics for eliminating biofilms and/or limiting their occurrence.

To prevent the formation of biofilms on surfaces, one method that has proven its efficacy is the use of antimicrobial coatings on these surfaces. Bacteria do not readily develop resistance to these antimicrobial coatings, which have a mechanical action and are not solely chemical.

Antimicrobial coatings may be obtained by functionalization of surfaces with molecules that have antimicrobial properties.

Patent application WO 2014/118 779, for example, describes a method for functionalizing surfaces with difunctional molecules comprising a catechol anchor and a peptide part comprising a difluorophenylalanine dipeptide. The purpose of functionalization is to confer antifouling properties to the surface, notably preventing the formation of biofilms on the surface.

The catechol anchor allows grafting onto surfaces by adsorption. Such grafting is less robust than covalent grafting.

It would be advantageous to have novel processes for functionalization of surfaces with peptide conjugates, said processes providing effective anchoring of the peptide conjugates on the surfaces. Peptide conjugates should advantageously be adaptable so that they can be used in a wide range of applications and with a wide range of peptides. Thus, it would be advantageous to be able to use functionalization processes with peptide conjugates in which the nature of the peptide, its distance from the anchor head, and its position of attachment to the anchor head, notably, can be controlled and/or adapted.

In this context, the inventors of the present invention have demonstrated that it is possible to efficiently functionalize solid support surfaces with peptide conjugates including a benzophenone-type anchor head, a spacer arm and a peptide fragment that may be attached to the spacer arm at different points in its structure. This functionalization is particularly suitable for plastic and/or textile supports.

### SUMMARY OF THE INVENTION

Thus, the present invention relates to a process for functionalization of at least one surface of a solid support, which involves contacting at least said surface of said solid support with at least one peptide conjugate of formula (I)

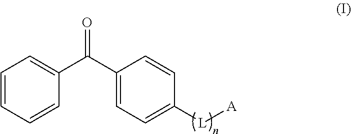

in which L is a spacer arm,
n is 0 or 1, preferably 1, and
A is a peptide fragment,
under conditions suitable for obtaining the anchoring of the at least one peptide conjugate of formula (I) to the surface of the support.

In one embodiment, A is a peptide fragment comprising from 2 to 80 amino acids, preferably from 3 to 30 amino acids, in particular from 7 to 20 amino acids.

In one embodiment, A is a peptide fragment chosen from the group consisting of an antibiotic peptide, an antimicrobial peptide, an antifungal peptide, an anti-inflammatory peptide, a catalytic peptide, a biological receptor ligand peptide, an antibody and an enzyme-inhibiting peptide, preferably an antimicrobial peptide, or a fragment thereof.

In one embodiment, the peptide fragment is an antimicrobial peptide chosen from the group consisting of the peptides H—(RF)₄—NH₂ (SEQ ID NO: 2), H—(RI)₄—NH₂ (SEQ ID NO: 4) and H—(R)₂-Palm.

In one embodiment, L is a saturated or unsaturated aliphatic hydrocarbon-based chain comprising from 1 to 10 carbon atoms, optionally interrupted or terminating with at least one selected from a heteroatom, notably O or S, an aryl group, a C═O group, an SO₂ group, a group NRᵢ, in which Rᵢ is chosen from a hydrogen atom, an aliphatic hydrocarbon-based radical comprising from 1 to 6 carbon atoms, a benzyl radical and a phenethyl radical, said chain possibly being unsubstituted or substituted. Preferably, L is an aliphatic carbon chain of formula (II)

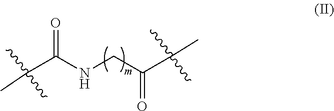

in which m is an integer from 1 to 10, preferably from 4 to 6, in particular 5.

In one embodiment, the solid support is chosen from the group consisting of a plastic support and a textile support.

In one embodiment, the functionalization process involves the following steps:
a. contacting at least said surface of said solid support with a solution or suspension of at least one peptide conjugate of formula (I) as defined above in a solvent, the contacting preferably being performed by soaking, spraying and/or incubation;
b. irradiating, notably via UV irradiation, the surface in contact with the solution or suspension of the at least one peptide conjugate of formula (I) for a period of time suitable to obtain the grafting of all or some of the peptide conjugate of formula (I) onto the surface; and
c. rinsing all or some of the at least one surface of the solid support with a solvent.

In one embodiment, the process further comprises at least one step chosen from the following steps:
a step (i), prior to step (a) of contacting, of activating the at least one surface, preferably performed by thermal activation, chemical activation and/or irradiation;
a step (i′), prior to step (a) of contacting, of cleaning the at least one surface;
a step (ii), after the rinsing step (c), of centrifugation;
a step (iii), after step (c) and, if it is present, after the centrifugation step (ii), of aging, preferably performed by aging under vacuum and/or by heating.

The present invention also relates to a solid support, at least one surface of which is coated with at least one peptide conjugate, that may be obtained, and preferably that is obtained, via the functionalization process according to the invention.

The present invention also relates to the use of a solid support according to the invention for the manufacture of nanoparticles for diagnostics, for the functionalization of Elisa plates, for the manufacture of antifouling surfaces, for the manufacture of medical devices, and/or for the manufacture of technical textiles.

### BRIEF DESCRIPTION OF THE DRAWINGS

FIG. 1 represents the percentages of inhibition of the two bacteria Escherichia coli and Staphylococcus aureus obtained by depositing peptide conjugates according to the invention onto polycarbonate plates. Top left: conjugate 1a; top right: conjugate 1b; bottom left: conjugate 1a; bottom right: conjugate 1b.

FIG. 2 is a photo showing the fluorescence of the three wells of the plate functionalized in Example 3.

### DETAILED DESCRIPTION

### Definitions

According to the invention, a “spacer arm” is a chemical group comprising at least one atom, preferably 1 to 10 atoms. Preferably, a spacer arm is a saturated or unsaturated aliphatic hydrocarbon-based chain comprising from 1 to 10 carbon atoms, optionally interrupted and/or terminating with at least one selected from a heteroatom, notably O or S, an aryl group, a C═O group, an SO₂ group, a group NRᵢ in which Rᵢ may notably be chosen from a hydrogen atom, an aliphatic hydrocarbon-based radical comprising from 1 to 6 carbon atoms, a benzyl radical and a phenethyl radical, said chain possibly being unsubstituted or substituted with one or more radicals, said radicals notably being chosen from halogen atoms (F, Cl, Br or I), a hydroxyl group, saturated aliphatic hydrocarbon-based chains comprising from 1 to 4 carbon atoms, benzyl radicals, phenethyl radicals, polyethylene oxide (PEG) radicals and polyalanine radicals.

A “peptide fragment” is a chain of at least two amino acids linked together by peptide and/or pseudopeptide bonds. Preferably, the peptide fragment comprises from 2 to 80 amino acids, preferably from 2 to 40 amino acids, preferably from 3 to 40 amino acids, preferably from 3 to 30 amino acids, preferably from 4 to 30 amino acids, preferably from 4 to 20 amino acids, in particular from 7 to 20 amino acids.

An “amino acid” is a molecule comprising at least one carboxylic acid function (COOH) and one amine function, and at least one carbon atom connecting this carboxylic acid function and this amine function. The amino acids used in the present invention may notably be natural and/or synthetic amino acids.

Natural amino acids notably include the following amino acids: glycine (Gly, G), alanine (Ala, A), valine (Val, V), leucine (Leu, L), isoleucine (Ile, I), serine (Ser, S), threonine (Thr, T), phenylalanine (Phe, F), tyrosine (Tyr, Y), tryptophan (Trp, W), cysteine (Cys, C), methionine (Met, M), proline (Pro, P), aspartic acid (Asp, D), asparagine (Asn, N), glutamine (Gln, Q), glutamic acid (Glu, E), histidine (His, H), arginine (Arg, R), and lysine (Lys, K). The preferred natural amino acids according to the present invention are amino acids of the L-series.

Synthetic amino acids are all non-natural amino acids. They notably include the following amino acids: beta-alanine, allylglycine, tert-leucine, norleucine, 3-aminoadipic acid, 2-aminobenzoic acid, 3-aminobenzoic acid, 4-aminobenzoic acid, 2-aminobutanoic acid, 4-amino-1-carboxymethylpiperidine, 1-amino-1-cyclobutanecarboxylic acid, 4-aminocyclohexaneacetic acid, 1-amino-1-cyclohexanecarboxylic acid, (1R,2R)-2-aminocyclohexanecarboxylic acid, (1R,2S)-2-aminocyclohexanecarboxylic acid, (1S,2R)-2-aminocyclohexanecarboxylic acid, (1S,2S)-2-aminocyclohexanecarboxylic acid, 3-aminocyclohexanecarboxylic acid, 4-aminocyclohexanecarboxylic acid, (1R,2R)-2-aminocyclopentanecarboxylic acid, (1R,2S)-2-aminocyclopentanecarboxylic acid, 1-amino-1-cyclopentanecarboxylic acid, 1-amino-1-cyclopropanecarboxylic acid, 3-aminomethylbenzoic acid, 4-aminomethylbenzoic acid, 2-aminobutanoic acid, 4-aminobutanoic acid, 6-aminohexanoic acid, 1-aminoindane-1-carboxylic acid, 2-aminoisobutyric acid, 4-aminomethylphenylacetic acid, 4-aminophenylacetic acid, 3-amino-2-naphthoic acid, 4-aminophenylbutanoic acid, 4-amino-5-(3-indolyl) pentanoic acid, (4R,5S)-4-amino-5-methylheptanoic acid, (R)-4-amino-5-methylhexanoic acid, (R)-4-amino-6-methylthiohexanoic acid, (S)-4-aminopentanoic acid, (R)-4-amino-5-phenylpentanoic acid, 4-aminophenylpropionic acid, (R)-4-aminopimeric acid, (4R,5R)-4-amino-5-hydroxyhexanoic acid, (R)-4-amino-5-hydroxypentanoic acid, (R)-4-amino-5-(p-hydroxyphenyl) pentanoic acid, 8-aminooctanoic acid, (2S,4R)-4-aminopyrrolidine-2-carboxylic acid, (2S,4S)-4-aminopyrrolidine-2-carboxylic acid, azetidine-2-carboxylic acid, (2S,4R)-4-benzyl-pyrrolidine-2-carboxylic acid, (S)-4,8-diaminooctanoic acid, tert-butylglycine, gamma-carboxyglutamate, beta-cyclohexylalanine, citrulline, 2,3-diaminopropionic acid, hippuric acid, homocyclohexylalanine, moleucine, homophenylalanine, 4-hydroxyproline, indoline-2-carboxylic acid, isonipecotic acid, α-methylalanine, naphthylalanine, nicopetic acid, norvaline, octahydroindole-2-carboxylic acid, ornithine, penicillamine, phenylglycine, 4-phenylpyrrolidine-2-carboxylic acid, propargylglycine, 3-pyridinylalanine, 4-pyridinylalanine, 1-pyrrolidine-3-carboxylic acid, sarcosine, statins, tetrahydroisoquinoline-1-carboxylic acid, 1,2,3,4-tetrahydroisoquinoline-3-carboxylic acid, tranexamic acid, 4,4-difluoroprolin, 4-fluoroproline, α-(3,4-difluorobenzyl) proline, gamma-(3,4-difluorobenzyl) proline, α-(trifluoromethyl)phenylalanine, hexafluoroleucine, 5,5,5-trifluoroleucine, 6,6,6-trifluoronorleucine, 2-(trifluoromethyl) leucine, 2-(trifluoromethyl) norleucine, 4,4,4-trifluorovaline, 4,4,4,4′,4′,4′-hexafluorovaline, pentafluorophenylalanine, 2,3-2,5-difluorophenylalanine, 2,6-difluorophenylalanine, 2,4-difluorophenylalanine, difluorophenylalanine, 3,4-difluorophenylalanine, 3,5-difluorophenylalanine, 3,3-difluoro-3-(4-fluorophenyl) alanine, 2,3-difluorophenylglycine, 2,4-difluorophenylglycine, 2,5-difluorophenylglycine, 3,4-difluorophenylglycine, 4,4-difluoroethylglycine, 4,4,4-trifluoroethylglycine, 4-fluorotryptophan, 5-fluorotryptophan, 6-fluorotryptophan, 5-methyltryptophan, S-tritylcysteine, selenocysteine, selenomethionine, ethionine, P-(2-thienyl) alanine, beta-chloroalanine, thiazolylalanine, triazolalanine, p-fluorophenylalanine, o-fluorophenylalanine, m-fluorophenylalanine, dihydroxyphenylalanine, 2,5-dihydrophenylalanine, thioproline, pipecolic acid, canavanine, indospicine, 3,4-dehydroproline, histidinol and hexafluoronorleucine, and the like or derivatives thereof.

The term “side chain” represents the fragment borne by the a carbon of an amino acid. For example, the side chains of natural amino acids such as glycine, valine, alanine, and aspartic acid correspond to the hydrogen atom, isopropyl, methyl and CH₂COOH groups, respectively. The side chains of other amino acids may be included in the definition of an amino acid side chain, such as those of the following amino acids: 4-aminotetrahydropyran-4-carboxylic acid, allylglycine, diaminobutyric acid, diaminopropionic acid, aminoserine, aminobutyric acid, aminobutylglycine, phenylglycine, 4-chlorophenylalanine, 4-fluorophenylalanine, 4-nitrophenylalanine, citrulline, cyclohexylalanine, thienylalanine, and the like. The side chains of the amino acids may be protected with protective groups (P) and more particularly N-protectors, O-protectors, or S-protectors when these chains contain the corresponding heteroatoms. The protection of certain reactive functions of peptides is mandatory during the synthesis of said peptides.

“Protective groups (P)” are groups known to those skilled in the art. These protective groups and their use are described, for example, in publications such as Greene, “Protective Groups in Organic Synthesis,” Wiley, New York, 2007, 4th edition; Harrison et al. “Compendium of Synthetic Organic Methods,” Vol. 1 to 8 (J. Wiley & Sons, 1971 to 1996). Also, peptide synthesis techniques are described in Paul Lloyd-Williams, Fernando Albericio, Ernest Giralt, “Chemical Approaches to the Synthesis of Peptides and Proteins,” CRC Press, 1997 or Houben-Weyl, “Methods of Organic Chemistry, Synthesis of Peptides and Peptidomimetics,” Vol. E 22a, Vol. E 22b, Vol. E 22c, Vol. E 22d., M. Goodmann Ed., Georg Thieme Verlag, 2002. Protective groups borne by a nitrogen atom will be denoted as N-protective groups; protective groups borne by a sulfur atom will be denoted as S-protective groups, and protective groups borne by an oxygen atom will be denoted as O-protective groups. For example, a hydroxyl group can be protected with a trityl group, or a carboxylic acid can be protected in the form of a tert-butyl ester. In the case of solid-phase synthesis, the resin serves as a protective group for the C-terminal carboxylic function. The amine group (i.e. “a amine”) of the amino acid may be protected, for example, with a tert-butoxycarbonyl (Boc-) group or a 9-fluorenyl-methyloxycarbonyl (Fmoc-) group. The protection is performed according to the processes known in the art. In the case of protecting the functional groups of natural amino acids, the amino acids obtained are synthetic until the protective group(s) are removed, thus releasing the “natural” amino acid. The removal of the protective group(s) is also performed according to the processes known in the art.

A “solid support” or “substrate” is an object of which at least one surface is solid, and at least that surface can be functionalized with the invention.

The term “plastic” denotes a support or surface comprising a mixture containing a base material that is a polymer or a mixture of polymers. The plastic support or surface may have been obtained by molding, extrusion, or shaping, preferably under heat and pressure, of said mixture comprising a polymer.

The term “textile” denotes an assembly of threads or fibers, advantageously bonded together, forming a solid and insoluble entity. Thus, and in an appropriate manner, the textile may be a fabric, advantageously obtained by weaving or knitting threads, or a nonwoven fabric obtained by assembling fibers. The textile material may notably be a fabric or a synthetic material such as a plastic material. The term “textile” notably includes medical textiles, civil textiles, technical textiles and filtration membranes.

The term “functionalization” or “anchoring” denotes the covalent or non-covalent attachment of at least one peptide conjugate to the surface of the solid support, preferably by means of the attachment, notably the covalent attachment, of the benzophenone unit of the conjugate to said surface.

The term “degree of functionalization” denotes the amount or density of peptide conjugate of formula (I) that is anchored to the surface of the solid support functionalized according to the invention.

### Peptide Conjugate of Formula (I)

The peptide conjugate used in the functionalization process according to the invention is a conjugate of formula (I)

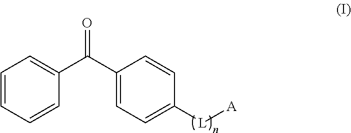

in which Lis a spacer arm, n is equal to 0 or 1, and A is a peptide fragment.

In formula (I), the spacer arm L is present (n=1) or absent (n=0); preferably, it is present (n=1). The presence of this spacer arm can notably distance the steric hindrance from the surface when the peptide fragment is bulky, for example when it comprises side chains.

The nature of the spacer arm may vary to a large extent. In certain embodiments, L is a saturated or unsaturated aliphatic hydrocarbon-based chain comprising from 1 to 10 carbon atoms, optionally interrupted or terminating with at least one selected from a heteroatom, notably O or S, an aryl group, a C═O group, an SO₂ group, a group NRᵢ, in which Rᵢ is chosen from a hydrogen atom, an aliphatic hydrocarbon-based radical comprising from 1 to 6 carbon atoms, a benzyl radical or a phenethyl radical, said chain possibly being unsubstituted or substituted.

In certain embodiments, L is an aliphatic carbon chain of formula (II)

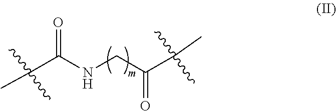

in which m is an integer from 1 to 10, preferably from 4 to 6, in particular 5.

The ends of the spacer arm L that are attached, respectively, to the benzophenone and to the peptide fragment A are typically formed of functional groups allowing the attachment of the different species by chemical reactions that are well known in the art, such as peptide bond formation reactions.

In one embodiment, the bond between the spacer arm L and the peptide fragment A is at the N-terminal end of the peptide fragment A. In another embodiment, the bond between the spacer arm L and the peptide fragment A is at the C-terminal end of the peptide fragment A. In another embodiment, the bond between the spacer arm L and the peptide fragment A is at a side chain of the peptide fragment A.

In one embodiment, the peptide conjugate comprises a single peptide fragment and a single benzophenone head. In other embodiments, the peptide conjugate may comprise a single peptide fragment and several benzophenone heads, for example 2, 3, 4, or 5 benzophenone heads preferably linked at different positions on the peptide fragment.

The peptide fragment may comprise from 2 to 80 amino acids, preferably from 2 to 40 amino acids, preferably from 3 to 40 amino acids, preferably from 3 to 30 amino acids, preferably from 4 to 30 amino acids, preferably from 4 to 20 amino acids, in particular from 7 to 20 amino acids. In one embodiment, peptide fragment A is not the GRGDSP fragment (SEQ ID NO: 1).

In one embodiment, peptide fragment A is not the RGD fragment. In one embodiment, peptide fragment A is neither the GRGDSP fragment (SEQ ID NO: 1) nor the RGD fragment.

In one embodiment, peptide fragment A does not comprise any amino acid that can react with benzophenone, such as methionine and/or tryptophan.

In one embodiment, peptide fragment A is an unmodified peptide. In another embodiment, the peptide fragment is a modified peptide, for example a peptide in which the end(s) not linked to the spacer arm are substituted and/or protected with a protective group. For example, the COOH end of the peptide may be substituted with a fatty acid, such as palmitic acid.

In one embodiment, peptide fragment A is chosen from the group consisting of a linear natural peptide strand, a linear synthetic peptide strand, a linear protected natural peptide strand, a linear protected synthetic peptide strand, a linear natural pseudopeptide strand, a linear synthetic pseudopeptide strand, a linear protected natural pseudopeptide strand, and a linear protected synthetic pseudopeptide strand. In another embodiment, peptide fragment A is chosen from the group consisting of a cyclic natural peptide fragment, a cyclic synthetic peptide fragment, a cyclic protected natural peptide fragment, a cyclic synthetic peptide fragment, a cyclic natural pseudopeptide fragment, a cyclic synthetic pseudopeptide fragment, a protected cyclic natural pseudopeptide fragment, and a protected cyclic synthetic pseudopeptide fragment.

The peptide fragment A of the peptide conjugate of formula (I) may comprise one or more peptides, peptide strands, and/or peptide fragments as described in the present description.

In one embodiment, peptide fragment A is chosen from the group consisting of the peptides H—(RF)₄—NH₂ (SEQ ID NO: 2), H—(RI)₄—NH₂ (SEQ ID NO: 4) and H—(R)₂-Palm, where Palm denotes modification of the terminal arginine with palmitic acid.

Advantageously, peptide fragment A has a particular property, notably particular chemical or biological activity. Thus, peptide fragment A may be chosen from the group consisting of an antibiotic peptide, an antimicrobial peptide, an antifungal peptide, an anti-inflammatory peptide, a catalytic peptide, a biological receptor ligand peptide, an antibody and an enzyme-inhibiting peptide. Preferably, peptide fragment A is chosen from the group consisting of an antibiotic peptide, an antimicrobial peptide, an antifungal peptide, an anti-inflammatory peptide and a catalytic peptide. In particular, peptide fragment A is chosen from the group consisting of an antibiotic peptide, an antimicrobial peptide and an antifungal peptide.

In one embodiment, peptide fragment A is an antimicrobial peptide, preferably a cationic antimicrobial peptide, with an overall positive charge and including at least one hydrophobic residue.

The particular property of peptide fragment A is advantageously not affected by its grafting to the spacer arm and to the benzophenone head: this particular property is thus also a particular property of the corresponding peptide conjugate. Similarly, the particular property of the peptide conjugate is advantageously not affected by its anchoring to the surface of the solid support: this particular property is thus also a particular property of the functionalized surface of the corresponding solid support.

In one embodiment, peptide fragment A is an antimicrobial peptide chosen from the group consisting of the peptides H—(RF)₄—NH₂ (SEQ ID NO: 2), H—(RI)₄—NH₂ (SEQ ID NO: 4) and H—(R)₂-Palm, where Palm denotes modification of the terminal arginine with palmitic acid.

In one embodiment, the peptide conjugate is chosen from the group consisting of the conjugate defined by SEQ ID NO: 3 and the conjugate defined by SEQ ID NO: 5.

The peptide conjugates according to the invention may be synthesized via any suitable technique known in the art, notably by successive couplings of the spacer arm with benzophenone and with peptide fragment A.

The peptide conjugate and peptide fragment A may notably be synthesized using conventional peptide synthesis techniques. Peptide synthesis is conventionally performed by activating the carboxylic function of an amino acid or amino acid chain using a coupling agent. This activated acid is brought into contact with an amino acid or amino acid chain whose terminal amine is unprotected, resulting in the formation of an amide bond, also referred to as a peptide bond.

The coupling conditions and coupling agents used are well known to those skilled in the art and are described, for example, in publications such as Greene, “Protective Groups in Organic Synthesis,” Wiley, New York, 2007, 4th edition; Harrison et al. “Compendium of Synthetic Organic Methods”, Vol. 1 to 8 (J. Wiley & Sons, 1971 to 1996). In addition, peptide synthesis techniques are described in Paul Lloyd-Williams, Fernando Albericio, Ernest Giralt, “Chemical Approaches to the Synthesis of Peptides and Proteins,” CRC Press, 1997 or Houben-Weyl, “Methods of Organic Chemistry, Synthesis of Peptides and Peptidomimetics,” Vol. E 22a, Vol. E 22b, Vol. E 22c, Vol. E 22d., M. Goodmann Ed., Georg Thieme Verlag, 2002.

### Functionalization Process

The functionalization process according to the invention is applied to at least one surface of a solid support. In one embodiment, the surface functionalized via the process according to the invention is the entire surface of the solid support. In another preferred embodiment, the surface functionalized via the process according to the invention is only a portion of the entire surface of the solid support.

The solid support whose surface is functionalized according to the invention may be made of any material that can beneficiate from functionalization with the peptide conjugate of formula (I). It may notably be a plastic support or a textile support, preferably a plastic support.

In the case where the peptide fragment A of the peptide conjugate of formula (I) has a particular property, notably a particular chemical or biological activity, the functionalization of the at least one surface of the solid support allows said property to be conferred on the at least one surface of the support.

In one embodiment, the process is performed with a single peptide conjugate of formula (I). In other embodiments, the process is performed with at least two different peptide conjugates, either one after the other or as a mixture. For example, the process may be performed with at least two peptide conjugates including two different peptide fragments, or with at least two peptide conjugates including the same peptide fragment but different spacer arms, in particular spacer arms of different lengths.

The contacting of the at least one surface with the at least one peptide conjugate of formula (I) may be performed via any suitable technique. Notably, the contacting may be performed by dipping, spraying and/or incubating the at least one surface with a solution or suspension of the peptide conjugate of formula (I) in a solvent. The solvent may be an organic solvent, an inorganic solvent, or a mixture of such solvents. The solvent may notably be an alcohol, such as ethanol. In one embodiment, the peptide conjugate of formula (I) is soluble in the solvent used. In particular, the techniques of deposition by centrifugation (“spin coating”), deposition by dipping (“dip coating”), deposition by drops (“casting”), deposition by laminar stream, and deposition by spraying (“aerospray”) may be mentioned.

A person skilled in the art is capable of determining parameters such as the duration and/or temperature of the contacting, as a function notably of the nature of the surface of the solid support, the structure of the peptide conjugate of formula (I) and/or the desired degree of functionalization. For example, the duration of the contacting may correspond to the time required for the complete evaporation of the solvent in which the conjugate is dissolved and/or suspended.

The functionalization process may allow at least one layer, preferably a monolayer, of the peptide fragment(s) of the peptide conjugate(s) of formula (I) to be anchored to the surface of the solid support.

In one embodiment, the functionalization process according to the invention comprises the following steps, preferably in this order:
a. contacting at least said surface of said solid support with a solution or suspension of at least one peptide conjugate of formula (I) in a solvent, the contacting preferably being performed by dipping, spraying, and/or incubation;
b. irradiating the surface in contact with the solution or suspension of the at least one peptide conjugate of formula (I) for a period of time suitable to obtain the anchoring of all or some of the peptide conjugate of formula (I) to the surface; and
c. rinsing all or some of the at least one surface of the solid support with a solvent.

Step a. of contacting may be performed as described above for the step of contacting.

Step b. of irradiation notably allows the activation of the benzophenone head of the peptide conjugate of formula (I) and its anchoring on the surface of the solid support. Preferably, this involves irradiation with ultraviolet (UV) light. A person skilled in the art is capable of adjusting the irradiation parameters, such as the irradiation duration, intensity, and/or wavelength, as a function notably of the nature of the surface of the solid support, the structure of the peptide conjugate of formula (I), and/or the desired degree of functionalization. Step b. of irradiation may be performed either in the presence of the solvent of the solution or suspension contacted with the support in step a., or after evaporation or drying of all or some of the solvent, notably after evaporation or drying of all the solvent.

Step c. of rinsing notably allows the removal of excess peptide conjugate of formula (I) that has not anchored to the surface of the solid support on conclusion of the irradiation step b. Rinsing may be performed with one or more solvents, either simultaneously or one after the other. The solvent used in the rinsing step c. may be an organic solvent, an inorganic solvent, or a mixture of such solvents. The rinsing step may be performed one or more times, notably 2, 3, 4, 5 or 10 times, using the same solvent or different solvents. Preferably, step c. of rinsing allows the removal of all of the peptide conjugate of formula (I) that is not anchored to the surface on conclusion of step b.

In one embodiment, the functionalization process further involves, prior to the contacting step a., a step (i) of activation of the at least one surface. This activation step notably makes it possible to improve the subsequent anchoring of the peptide conjugate of formula (I) to the surface, for example by increasing the degree of anchoring and/or increasing the degree of functionalization. The nature of the activation to be used depends on the nature of the surface of the solid support. This activation may notably be thermal activation, by exposure to a temperature above room temperature (15 to 25° C.), chemical activation, by contacting with a chemical agent allowing activation, and/or activation by irradiation.

In one embodiment, the functionalization process further involves, prior to the contacting step a., a step (i′) of cleaning the at least one surface. The purpose of this step is to remove any substance present on the surface that could prevent or weaken subsequent functionalization with the peptide conjugate(s). The cleaning step (i′) may be performed, for example, using a solvent such as an alcohol, notably ethanol or isopropanol.

In one embodiment, the functionalization process further involves, after the rinsing step c., a centrifugation step (ii), preferably also comprising the separation of the pellet and the centrifugation supernatant, the solid support being in the centrifugation pellet.

In one embodiment, the functionalization process further involves, after the rinsing step c. and after the centrifugation step (ii) if it is present, an aging step (iii). Step (iii) may notably be a step of aging under reduced pressure (under vacuum) and/or a step of aging by heating to a temperature above room temperature. In one embodiment, step (iii) involves aging under reduced pressure and then aging by heating.

### Solid Support

Another subject of the present invention is a solid support of which at least one surface is coated with at least one peptide conjugate, that may be obtained, and preferably that is obtained, via the functionalization process according to the invention.

In certain embodiments, only one surface of the solid support is functionalized via a functionalization process according to the invention. In other embodiments, at least two surfaces of the solid support, preferably all the surfaces of the solid support, are functionalized via a functionalization process according to the invention.

In other words, in certain embodiments, only a portion of the surface of the solid support is functionalized via a functionalization process according to the invention. Thus, for example, 100% of the surface of the solid support is functionalized via a functionalization process according to the invention. In other embodiments, less than 100%, less than 90%, less than 80%, less than 70%, less than 60%, less than 50%, less than 40%, less than 30%, less than 20%, less than 10%, or less than 5% of the surface of the solid support is functionalized via a functionalization process according to the invention.

### Use of the Solid Support

The solid support according to the invention and/or the solid support that may be obtained, and preferably that is obtained, via a functionalization process according to the invention, may be used in a wide variety of applications.

Among the possible uses, mention may be made of the manufacture of nanoparticles for diagnostics, the functionalization of Elisa plates, the manufacture of antifouling surfaces, the manufacture of medical devices, and/or the manufacture of technical textiles.

Thus, a final subject of the invention is the use of a solid support according to the invention, a solid support that is obtained or that may be obtained via a functionalization process according to the invention, and/or a functionalization process according to the invention, for the manufacture of nanoparticles for diagnostics, for the functionalization of Elisa plates, for the manufacture of antifouling surfaces, for the manufacture of medical devices, and/or for the manufacture of technical textiles.

The examples that follow illustrate the invention in greater detail and should be considered as being illustrative and without implied limitation of the invention.

### EXAMPLES

### Preparation of Peptide-Substituted Monomers

The peptides are synthesized on a solid support using a Symphony X peptide synthesizer (Protein Technologies, Inc., USA) in an Fmoc/tert-butyl strategy using nitrogen sparging as the stirring method for the coupling and deprotection cycles of the Fmoc groups in the N-terminal position or in solution in a Boc/Bzl strategy.

The syntheses are performed on a scale of 0.25 mmol on Fmoc-Rink-Amide polystyrene resin (481 mg/0.52 mmol/g) or 2-chlorotrityl chloride (892 mg, 0.28 mmol/g). The standard deprotection-coupling cycle for each residue comprises six steps: Washing the resin with 5 mL of dimethylformamide DMF (3×30 sec). Deprotection of the Fmoc group with 5 mL of 20% piperidine in DMF (3× 3 min). Washing of the resin with 5 mL of DMF (3×30 sec). Coupling of the Fmoc-AA residue for 60 min using HATU (1-[bis(dimethylamino)methylene]-1H-1,2,3-triazolo[4,5-b]pyridinium 3-oxide hexafluorophosphate) and N,N-diisopropylethylamine DIEA as the coupling agent. At the end of the coupling cycle, a capping step is performed with 5 mL of 10% acetic anhydride Ac₂O in DMF for 7 min, and the resin is then washed with 5 mL of DMF (3× 30 sec).

The resin (Fmoc-Rink-Amide) cleavage is performed for 2× 60 minutes in a cleavage cocktail (95/2.5/2.5 trifluoroacetic acid TFA/triisopropylsilane TIS/water H₂O) for the unprotected sequences and 2× 60 minutes in a cleavage cocktail (10/20/70 acetic acid AcOH/2,2,2-trifluoroethanol TFE/dichloromethane DCM) for protected sequences (2-chlorotrityl chloride resin). Once the resin has been filtered off, the solution is evaporated under vacuum and the peptide is precipitated from diethyl ether. The precipitated peptide is centrifuged (3500 RCF) and the supernatant is removed (×3). The peptide in the form of TFA salts is then dissolved in a mixture of water H₂O/acetonitrile ACN before being frozen and freeze-dried.

The peptides are analyzed by UPLC chromatography and ESI-MS by mass spectrometry, equipped with a BEH C18 (Waters) column, 150×2.1 mm (150× 2.1 mm) (flow rate: 0.6 ml/min). Solvents A and B are 0.1% TFA in water and 0.1% TFA in acetonitrile. Peptide purification is performed on a Waters HPLC 4000 apparatus equipped with a UV 486 detector and a Vydac Denali 10 μm C18 120 Å column (310× 25 mm) at a flow rate of 50 mL/min. The solvents used are 0.1% TFA in water (buffer A) and 0.1% TFA in acetonitrile (buffer B).

### Example 1: Synthesis of Peptide Conjugates According to the Invention

### Example 1a. Peptide Conjugate Comprising the Peptide H—(RF)₄—NH₂ (SEQ ID NO: 2)

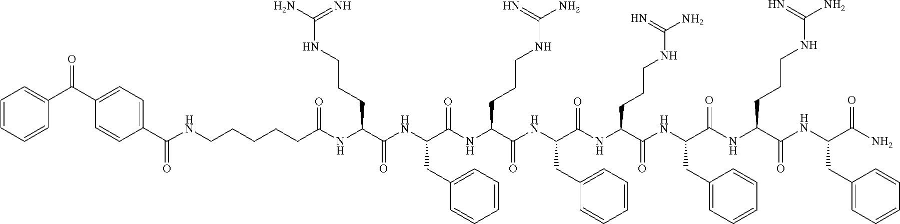 [SMILES: N=C(N)NCCC[C@H](NC(=O)CCCCCNC(=O)c1ccc(C(=O)c2ccccc2)cc1)C(=O)N[C@@H](Cc1ccccc1)C(=O)N[C@@H](CCCNC(=N)N)C(=O)N[C@@H](Cc1ccccc1)C(=O)N[C@@H](CCCNC(=N)N)C(=O)N[C@@H](Cc1ccccc1)C(=O)N[C@@H](CCCNC(=N)N)C(=O)N[C@@H](Cc1ccccc1)C(N)=O]

The antibacterial peptide H—(RF)₄—NH₂ (SEQ ID NO: 2) was synthesized on Rink Amide resin (load 0.52 mmol/g, synthesis scale: 0.25 mmol) using the Fmoc/tBu strategy. Each coupling was followed by N-terminal deprotection of the Fmoc group. The peptide was then functionalized on the support after introduction of a spacer (or “linker”). The Fmoc-Ahx-OH spacer (6-aminohexanoic acid protected with an Fmoc group) was introduced in DMF in the presence of three equivalents of HATU and six equivalents of DIEA, and the resin was washed (3×DMF, 1×methanol MeOH, and 1×dichloromethane DCM). 4-Benzoylbenzoic acid was coupled in DMF in the presence of three equivalents of HATU and six equivalents of DIEA, and the resin was washed (3×DMF, 1×MeOH, and 1×DCM). The resin was then cleaved in a 50/47.5/2.5 DCM/TFA/H₂O mixture for 2× 1 hour. The cleavage solution was concentrated under reduced pressure, and the antibacterial anchor peptide was then precipitated from diethyl ether and finally purified by preparative HPLC (Vydac Denali 10 μm C18 120 Å column (310× 25 mm) for its use, using a gradient from 20% to 60% of buffer B over 40 min.).

The conjugate obtained has the formula shown above and corresponds to SEQ ID NO: 3. ESI-MS (m/z): [M+H]⁺ theoretical C₇₅H₁₀₃N₂₂O₁₄: 1552.87, experimental: 1552.98 HPLC (retention time, min): 3.73

### Example 1b: Peptide Conjugate Comprising the Peptide H—(RI)₄—NH₂ (SEQ ID NO: 4)

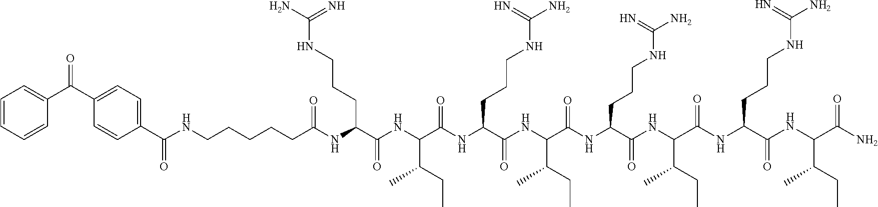 [SMILES: CC[C@H](C)C(NC(=O)[C@H](CCCNC(=N)N)NC(=O)C(NC(=O)[C@H](CCCNC(=N)N)NC(=O)C(NC(=O)[C@H](CCCNC(=N)N)NC(=O)C(NC(=O)[C@H](CCCNC(=N)N)NC(=O)CCCCCNC(=O)c1ccc(C(=O)c2ccccc2)cc1)[C@@H](C)CC)[C@@H](C)CC)[C@@H](C)CC)C(N)=O]

The antibacterial peptide H—(RI)₄—NH₂ (SEQ ID NO: 4) was synthesized on Rink Amide resin (load 0.52 mmol/g, synthesis scale: 0.25 mmol) using the Fmoc/tBu strategy. Each coupling was followed by N-terminal deprotection of the Fmoc group. The peptide was then functionalized on the support after introduction of a spacer (or “linker”). The Fmoc-Ahx-OH spacer was introduced in DMF in the presence of three equivalents of HATU and six equivalents of DIEA, and the resin was washed (3×DMF, 1×MeOH and 1×DCM). 4-Benzoylbenzoic acid was coupled in DMF in the presence of three equivalents of HATU and six equivalents of DIEA, and the resin was washed (3×DMF, 1×MeOH and 1×DCM). The resin was then cleaved in a 50/47.5/2.5 DCM/TFA/H₂O mixture for 2× 1 hour. The cleavage solution was concentrated under reduced pressure, and the antibacterial anchor peptide was then precipitated from diethyl ether and finally purified by preparative HPLC (Vydac Denali 10 μm C18 120 Å column (310× 25 mm) using a gradient from 20% to 60% of buffer B over 40 min.).

The conjugate obtained has the formula shown above and corresponds to SEQ ID NO: 5.

ESI-MS (m/z): [M+H]⁺ theoretical C₇₅H₁₀₃N₂₂O₁₄: 1416.80, experimental: 1496.98 HPLC (retention time, min): 3.63

### Example 1c: Peptide Conjugate Comprising the Peptide H—(R)₂-Palm

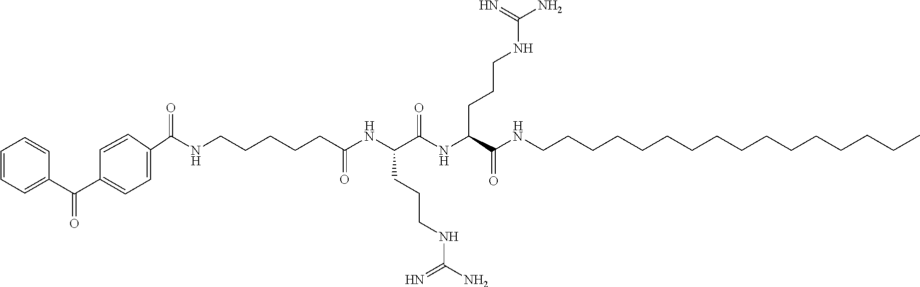 [SMILES: CCCCCCCCCCCCCCCCNC(=O)[C@H](CCCNC(=N)N)NC(=O)[C@H](CCCNC(=N)N)NC(=O)CCCCCNC(=O)c1ccc(C(=O)c2ccccc2)cc1]

The antibacterial peptide H—(R)₂-Palm was synthesized on Rink resin (load 0.52 mmol/g, synthesis scale: 0.25 mmol) previously functionalized with benzotriazole using an Fmoc/tBu strategy. Each coupling was followed by N-terminal deprotection of the Fmoc group. The Rink-amide resin was pretreated with piperidine (20% in DMF) and washed (3×DMF, 1×MeOH and 1×DCM). 4-Amino-3-nitrobenzoic acid was coupled in DMF in the presence of three equivalents of HATU and six equivalents of DIEA. The reaction was left stirring for 2 hours, and the resin was then washed (3×DMF, 1×MeOH and 1×DCM), capped with acetic anhydride (15 mL of a 10% v/v solution in DCM), and then washed (2×DCM, 3×DMF, 1×MeOH and 1×DCM). The reduction of the nitro group was performed in a solution of 5 g of SnCl₂·2H₂O and 900 μL of 1,8-diazabicyclo[5.4.0]undec-7-ene (DBU) per 10 mL of DMF, which was added to the resin while sparging with nitrogen for 10 minutes. The reaction was stirred for 15 hours, with the syringe open, and washed (3×DMF, 3×DCM).

The introduction of the peptide sequence —(R)₂— was performed using the Fmoc/tBu strategy. Each coupling was followed by N-terminal deprotection of the Fmoc group. The peptide was then functionalized on the support after introduction of a spacer (or “linker”). The Fmoc-Ahx-OH spacer was introduced in DMF in the presence of three equivalents of HATU and six equivalents of DIEA, and the resin was washed (3×DMF, 1×MeOH and 1×DCM). 4-Benzoylbenzoic acid was coupled in DMF in the presence of three equivalents of HATU and six equivalents of DIEA, and the resin was washed (3×DMF, 1×MeOH and 1×DCM). The resin was then treated with isoamyl nitrite (10 equivalents) for 90 minutes (washed with DCM×5). The nucleophile (4 equivalents/in this case hexadecylamine) in solution in DCM in the presence of DIEA (8 equivalents) was sparged with nitrogen for 20 minutes before the end of cyclization, and then immediately introduced onto the resin. The reaction was stirred, inducing cleavage of the peptide from the resin.

The peptide was then deprotected in a 50/47.5/2.5 DCM/TFA/H₂O mixture for 2×1 hour. The “deprotection” solution was concentrated under reduced pressure, and the antibacterial peptide was then precipitated from diethyl ether and finally purified by preparative HPLC (Vydac Denali 10 μm C18 120 Å column (310× 25 mm) using a gradient from 20% to 60% of buffer B over 40 min).

ESI-MS (m/z): [M+H]⁺ theoretical C₄₃H₇₅N₁₀O₈: 876.22 experimental: 876.19

HPLC (retention time, min): 5.63

### Example 2: Deposition of the Peptide Conjugates According to the Invention on a Substrate

The deposition of the three peptides obtained in Example 1 was performed by dip-coating, i.e. by dipping/removing the substrate at a constant speed in a solution of the anchor peptide in an ethanolic solution.

Each peptide was dissolved in an ethanolic solution (95%).

After a few minutes of stirring, a clear and stable solution was obtained. This solution was deposited onto a clean polycarbonate substrate by dipping. The dipped plastic plates were then dried and irradiated with UV light for 15 minutes (360 nm, 250 W). After irradiation, the plates were washed with sonication in water and ethanol baths (4×10 minutes).

### Example 3: Grafting of Benzophenone onto a Support

The fluorescent block dansyl-benzophenone, whose formula is shown below, was synthesized using conventional techniques.

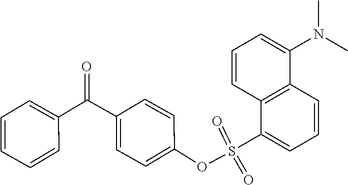 [SMILES: CN(C)c1cccc2c(S(=O)(=O)Oc3ccc(C(=O)c4ccccc4)cc3)cccc12]

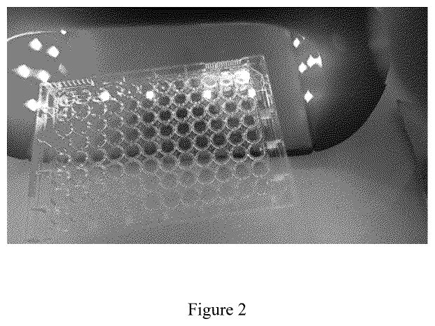

A 96-well polystyrene plate was first rinsed with distilled water and ethyl alcohol, and then dried. A 0.1M solution of dansyl-benzophenone compound was deposited in the first three wells. The plate was irradiated for 180 seconds under UV light at a power of 120 mW/cm². The plate was then rinsed with distilled water. FIG. 2 shows a photo of the plate, which has fluorescence in the three functionalized wells.

This result proves that the benzophenone head grafts onto the surface of the wells of the 96-well plate.

### Example 4: Determination of the Antibacterial Activity of Substrates Treated with the Antimicrobial Peptides According to the Invention

The antibacterial activity according to the standard ISO 22196:2011 is intended to evaluate the antimicrobial activity of plastic products or non-porous surfaces treated with antimicrobial agents. The method used in this study focuses solely on the evaluation of antibacterial activity. This study is intended to evaluate the antibacterial activity of polycarbonate materials grafted with peptides compared to the untreated reference according to the recommendations of the standard ISO 22196:2011 for a contact time of 24 hours.

Polycarbonate Material (6 cm×5 cm Rectangle)

Ideally, such a study uses 5 cm×5 cm samples on which a known concentration of the microorganism to be tested has been deposited. After incubation for 24 hours at 35° C., the amount of viable microorganisms is evaluated using the agar plate counting technique. The antibacterial activity of the formulation tested is defined by comparing the bacterial concentrations obtained between the treated material and the untreated material.

The study performed here was performed on samples in the form of 6 cm×5 cm rectangles in accordance with the standard. The tests were conducted on the bacterial strains prescribed by the standard and very frequently involved in infections, Escherichia coli ATCC 8739 and Staphylococcus aureus ATCC 6538P.

Each conjugate 1a, 1b, and 1c as synthesized in Example 1a was deposited on the surface of the polycarbonate plates by applying a 0.02 to 4 mg/mL solution.

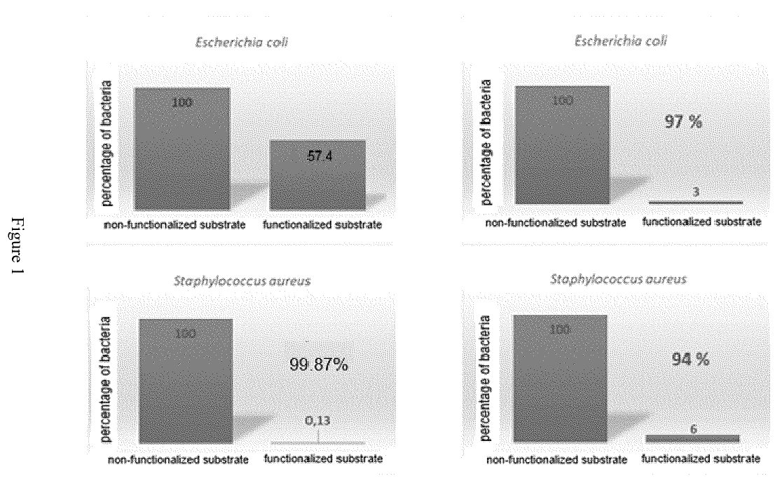

The results showed significant inhibition with a reduction in Staphylococcus aureus growth of up to 99.87% (Tables 1 and 2 and FIG. 1). Table 1 corresponds to Staphylococcus aureus, Table 2 corresponds to Escherichia coli.

In Tables 1 and 2, PC denotes polycarbonate. The peptide conjugates are denoted with reference to the example in which their synthesis is described (1a, 1b, and 1c).

TABLE 1

| | | Geometrical | Mean | | |
|---|---|---|---|---|---|
| | | mean | Log10 | R (log | % |
| Time | Reference | (CFU/cm²) | (CFU/cm²) | number) | reduction |
| 0 h | Negative | 1.62E+04 | 4.21 | | |
| | control | | | | |
| | PC blank | 1.42E+04 | 4.15 | | |
| 24 h | PC blank | 1.19E+04 | 4.07 | | |
| | PC/ | 1.54E+01 | 1.19 | 2.9 | 99.87 |
| | Peptide 1a | | | | |
| | PC/ | 7.08E+02 | 2.85 | 1.2 | 94.0 |
| | Peptide 1b | | | | |
| | PC/ | 6.34E+03 | 3.8 | 0.3 | 46.7 |
| | Peptide 1c | | | | |

TABLE 2

| | | Geometrical | Mean | | |
|---|---|---|---|---|---|
| | | mean | Log10 | R (log | % |
| Time | Reference | (CFU/cm²) | (CFU/cm²) | number) | reduction |
| 0 h | Negative | 1.35E+04 | 4.13 | | |
| | control | | | | |
| | PC blank | 1.42E+04 | 4.15 | | |
| 24 h | PC blank | 2.12E+06 | 6.33 | | |
| | PC/ | 1.22E+06 | 6.09 | 0.2 | 42.6 |
| | Peptide 1a | | | | |
| | PC/ | 6.31E+04 | 4.80 | 1.5 | 97.0 |
| | Peptide 1b | | | | |
| | PC/ | 1.24E+06 | 6.09 | 0.2 | 41.7 |
| | Peptide 1c | | | | |

FIG. 1 illustrates these results. Significant inhibition of both types of bacteria was obtained (up to 99.87% reduction in growth for Staphylococcus aureus, and up to 97.0% reduction in growth for Escherichia coli) for the polycarbonate samples functionalized according to the invention compared to the non-functionalized polycarbonate samples.

The antibacterial activity of the peptide conjugates 1a, 1b, and 1c is thus effectively transferred to the polycarbonate support onto which they are grafted.

1-10. (canceled)

11. A process for functionalization of at least one surface of a solid support, which involves contacting at least said surface of said solid support with at least one peptide conjugate of formula (I) 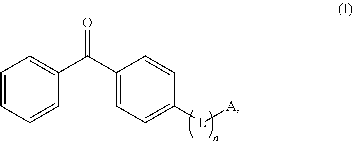
in which L is a spacer arm,
n is 0 or 1, and
A is a peptide fragment,
under conditions suitable for obtaining the anchoring of the at least one peptide conjugate of formula (I) to the surface of the support.

12. The process for functionalization of at least one surface of a solid support according to claim 11, wherein A is a peptide fragment comprising from 2 to 80 amino acids.

13. The process for functionalization of at least one surface of a solid support according to claim 11, wherein A is a peptide fragment selected from the group consisting of an antibiotic peptide, an antimicrobial peptide, an antifungal peptide, an anti-inflammatory peptide, a catalytic peptide, a biological receptor ligand peptide, an antibody and an enzyme-inhibiting peptide.

14. The process for functionalization of at least one surface of a solid support according to claim 11, wherein the peptide fragment is an antimicrobial peptide selected from the group consisting of the peptides H—(RF)₄—NH₂ (SEQ ID NO: 2), H—(RI)₄—NH₂ (SEQ ID NO: 4) and H—(R)₂-Palm.

15. The process for functionalization of at least one surface of a solid support according to claim 11, wherein L is a saturated or unsaturated aliphatic hydrocarbon-based chain comprising from 1 to 10 carbon atoms, optionally interrupted or terminating with at least one heteroatom, aryl group, C—O group, SO₂ group, group NRᵢ, in which Rᵢ is chosen from a hydrogen atom, an aliphatic hydrocarbon-based radical comprising from 1 to 6 carbon atoms, a benzyl radical or a phenethyl radical, said chain optionally being unsubstituted or substituted.

16. The process according to claim 15, wherein L is an aliphatic carbon chain of formula (II) 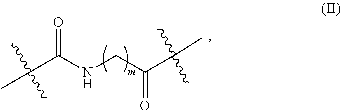
in which m is an integer from 1 to 10.

17. The process for functionalization of at least one surface of a solid support according to claim 11, wherein the solid support is selected from the group consisting of a plastic support and a textile support.

18. The process for functionalization of at least one surface of a solid support according to claim 11, which involves the following steps:
a) contacting at least said surface of said solid support with a solution or suspension of at least one peptide conjugate of formula (I) in a solvent, the contacting being performed by dipping, spraying and/or incubation;
b) irradiating, the surface in contact with the solution or suspension of at least one peptide conjugate of formula (I) for a period of time suitable for obtaining the grafting of all or some of the peptide conjugate of formula (I) onto the surface; and
c) rinsing all or some of the at least one surface of the solid support with a solvent.

19. The process according to claim 18, further comprising at least one of the following steps:
a step (i), prior to step (a) of contacting, of activating the at least one surface;
a step (i′), prior to step (a) of contacting, of cleaning the at least one surface;
a step (ii), after the rinsing step (c), of centrifugation; or
a step (iii), after step (c) and, if it is present, after the centrifugation step (ii), of aging.

20. The process according to claim 19, wherein said aging is performed by aging under vacuum and/or by heating.

21. The process according to claim 19, wherein said surface is activated by thermal activation, chemical activation and/or irradiation.

22. A solid support of which at least one surface is coated with at least one peptide via the functionalization process according to claim 11.

23. The solid support according to claim 22, wherein the solid support comprises nanoparticles for diagnostics, functionalized of ELISA plates, anti-fouling surfaces, medical devices, or technical textiles.
